(19) United States
(12) Patent Application Publication
(10) Pub. No.: US 2025/0387014 A1
(43) Pub. Date: Dec. 25, 2025

(54) MEDICAL DEVICES AND RELATED SYSTEMS AND METHODS

(71) Applicant: Boston Scientific Scimed, Inc., Maple Grove, MN (US)

(72) Inventors: John SUBASIC, Sutton, MA (US); Benjamin CLEVELAND, Bellingham, MA (US)

(73) Assignee: Boston Scientific Scimed, Inc., Maple Grove, MN (US)

(21) Appl. No.: 19/247,248

(22) Filed: Jun. 24, 2025

Related U.S. Application Data
(60) Provisional application No. 63/663,841, filed on Jun. 25, 2024.

(51) Int. Cl. A61B 1/00

(52) U.S. Cl. CPC A61B 1/00154; A61B 1/00135

(57) ABSTRACT

Medical devices and systems are described. The medical device may include a handle that includes a hub, a grip portion, and an arm. The hub may define a lumen having a proximal opening. The arm may extend proximally from the hub and may be coupled to a member slidable or rotatable relative to the arm. The grip portion may extend transverse to the lumen of the hub. The grip portion may be configured to receive a palm of a user's hand while a thumb of the user's hand controls sliding or rotation of the member.

### CROSS-REFERENCE TO RELATED APPLICATIONS

This application claims the benefit of priority of U.S. Provisional Application No. 63/663,841, filed on Jun. 25, 2024, which is incorporated by reference herein in its entirety.

### TECHNICAL FIELD

Aspects of the present disclosure generally relate to medical devices and medical systems. In particular, some aspects relate to medical devices and systems having a handle to control suction and movement of a shaft.

### BACKGROUND

Medical devices such as scopes and sheaths are often inserted into the body of a patient to perform a therapeutic and/or diagnostic procedure inside the body. Various features of the scope and/or sheath may assist in performing a therapeutic and/or diagnostic procedure inside the subject's body, e.g., including supply of irrigation or suction. Controlling distal and proximal movement of a shaft of the scope and controlling suction via a sheath surrounding the scope may require multiple hands and maintaining an uncomfortable position of the hands, and/or may require multiple operators. Such awkward positioning can lead to fatigue over time and potentially increase risk for the patient.

### SUMMARY

Each of the aspects disclosed herein may include one or more features described in connection with any of the other disclosed aspects.

The present disclosure includes a medical device comprising a handle that includes a hub, a grip portion, and an arm. The hub may define a lumen having a proximal opening. The arm may extend proximally from the hub and be coupled to a member slidable or rotatable relative to the arm. The grip portion may extend transverse to the lumen. The grip portion may be configured to receive a palm of a user's hand while a thumb of the user's hand may control sliding or rotation of the member.

In some examples, the member may be a cart slidable along a length of the arm or a wheel rotatable relative to the arm. For example, the member may be a cart slidable along the arm in a distal direction, e.g., by force applied to a proximal side of a projection of the cart, and in a proximal direction by a force applied to a distal side of the projection. Optionally, the arm may include a stop proximal to the cart that limits movement of the cart in the proximal direction. The arm may be biased in the proximal direction, e.g., the arm including a spring configured to bias the cart in the proximal direction. Additionally or alternatively, the arm may be rotatable relative to the hub about a pivot at a distal portion of the arm. In some examples, the member may include a wheel coupled to the arm, e.g., the wheel rotatable relative to the arm.

According to some aspects, the arm of the handle may be a first arm and the handle may include a second arm extending proximally from the hub and parallel to the first arm. In cases in which the member is a wheel, the wheel may be a first wheel coupled to a proximal end of the first arm. Optionally, the handle may include a second wheel coupled to a proximal end of the second arm.

According to some aspects, the medical device may include a sheath extending distally from the hub. The sheath may be in fluid communication with the lumen of the hub. The handle may include a suction channel in fluid communication with the sheath. An end of the suction channel may be configured to connect to a vacuum source. The handle may include an aperture in communication with the suction channel. The aperture may be configured to vent the suction channel. The suction channel may extend through the grip portion. The medical device may further include a shaft extending through the lumen of the hub and through the sheath. The shaft may be slidable along a length of the sheath. The hub may include a valve or adapter configured to form a seal with the shaft. The shaft may be a shaft of a scope, for example.

The present disclosure also includes a medical device comprising a handle and a sheath, e.g., the handle including a hub, a grip portion, and an arm. The hub may define a lumen that extends along a central longitudinal axis of the hub. The lumen may have a proximal opening. The grip portion may extend transverse to the central longitudinal axis. The arm may extend proximally from the hub. The arm may be coupled to a member, e.g., a cart, slidable along a length of the arm or a wheel rotatable relative to the arm. The sheath may extend distally from the hub and may be in fluid communication with the lumen of the hub. The grip portion may be configured to receive a palm of a user's hand while a thumb of the user's hand contacts the cart or the wheel.

In some examples, the handle may include a suction channel that extends through the grip portion and an aperture configured to vent the suction channel. The suction channel may be configured to connect to a vacuum source. The arm may be rotatable relative to the hub about a pivot at a distal portion of the arm. The grip portion may be substantially perpendicular to the central longitudinal axis of the hub.

The present disclosure also includes a medical device comprising a sheath and a handle including a hub, a grip portion, and an arm. The hub may define a lumen extending along a central longitudinal axis of the hub and may have a proximal opening configured to form a seal with a shaft, e.g., a shaft of a scope. The sheath may extend distally from the hub. The grip portion may extend transverse to the lumen. The arm may extend proximally from the hub and may be configured to pivot relative to the hub. The arm may be coupled to a member such as a cart or a wheel, the member slidable along a length of the arm or rotatable relative to the arm. The grip portion may be configured to receive a palm of a user's hand while a thumb of the user's hand controls sliding or rotation of the member.

### BRIEF DESCRIPTION OF THE FIGURES

The accompanying drawings, which are incorporated in and constitute a part of this application, illustrate exemplary aspects that, together with the written descriptions, serve to explain the principles of this disclosure. Each figure depicts one or more exemplary aspects according to this disclosure, as follows:

FIG. 1 depicts a perspective view of an exemplary medical system and medical device according to some aspects of the present disclosure.

FIGS. 2A-2B depict a side view of the handle of the medical device of FIG. 1 in a first configuration (FIG. 2A) and a second configuration (FIG. 2B).

FIGS. 3A-3B depict another exemplary medical system and medical device, illustrating a handle of the medical device in a first configuration (FIG. 3A) and in a second configuration (FIG. 3B).

FIGS. 4A-4B depict another exemplary medical system and medical device, illustrating a handle of the medical device in a first configuration (FIG. 4A) and in a second configuration (FIG. 4B).

FIGS. 5A-5C depict another exemplary medical system and medical device, wherein a handle of the medical device is in a first configuration in FIGS. 5A-5B and the handle of the medical device is in a second configuration in FIG. 5C.

### DETAILED DESCRIPTION

Particular aspects of the present disclosure are described in greater detail below. The terms and definitions provided herein control, if in conflict with terms and/or definitions incorporated by reference. The term “distal” refers to a portion farthest away from a user when introducing a device into a subject (e.g., patient). By contrast, the term “proximal” refers to a portion closest to the user when placing the device into the subject. Proximal and distal directions are labeled with arrows marked “P” and “D,” respectively, throughout various figures.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range.

Although scopes and ureteroscopes are referenced herein for illustration purposes, it will be appreciated that the disclosure encompasses any suitable medical device configured to allow an operator to access and view internal body anatomy of a subject and/or to deliver medical instruments, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device or medical device may be used in connection with the features described herein.

The medical systems described in this disclosure may include a medical device and optionally a scope (e.g., ureteroscope or endoscope) receivable within, and translatable along, a portion of the medical device. For example, at least of portion of the scope (e.g., a shaft) may be slidable relative to the medical device. The medical device may include a handle that comprises a hub, a grip portion, and an arm extending proximally from the hub, the arm having a member such as a cart or a wheel movable relative to the arm. The grip portion and the arm of the handle may have proximity to each other such that a user can grasp the grip portion with the palm of the user's hand while the thumb or other finger of the user's hand controls movement of the member relative to the arm.

The medical device also may include a sheath configured to receive a scope, e.g., the shaft of the scope. The shaft may be slidable relative to the handle and the sheath of the medical device. In some aspects, the medical device may be configured to control suction through the sheath. For example, the handle of the medical device may include an aperture in communication with a suction channel of the medical device, wherein the user may control suction by covering and uncovering the aperture with only one finger while the user grasps the grip portion. The medical device may include features configured to aid the user in moving the shaft of the scope relative to the sheath of the medical device. For example, the medical device may be configured so that the user may control sliding of the shaft relative to the sheath with the user's thumb or another digit as the user grasps the grip portion of the handle. Portions of the medical device may be configured to rotate radially outward relative to a central longitudinal axis of the medical device to facilitate movement of the scope relative to the medical device.

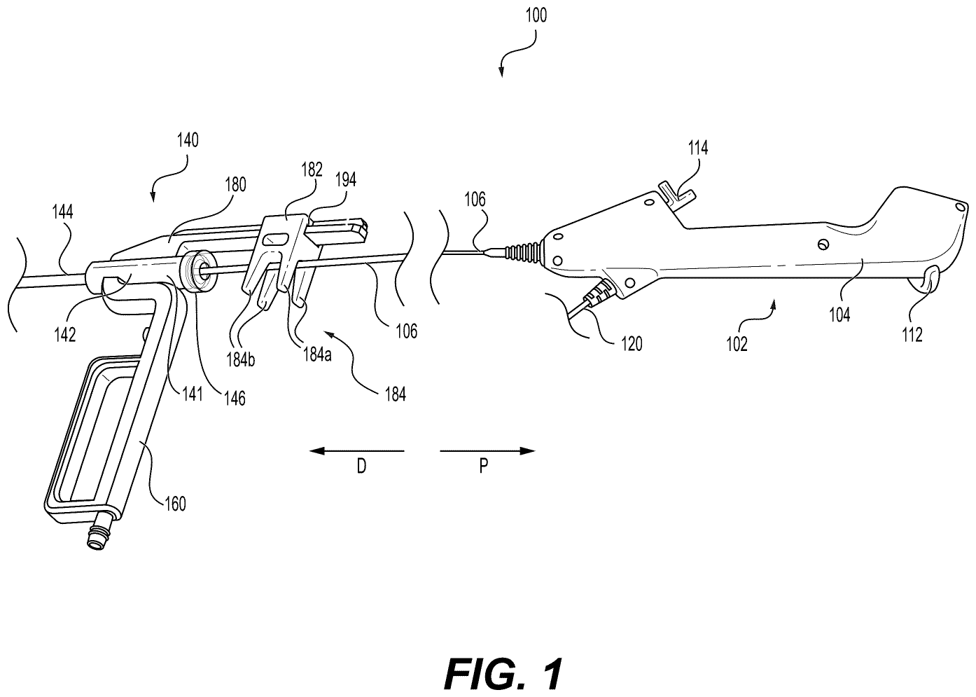

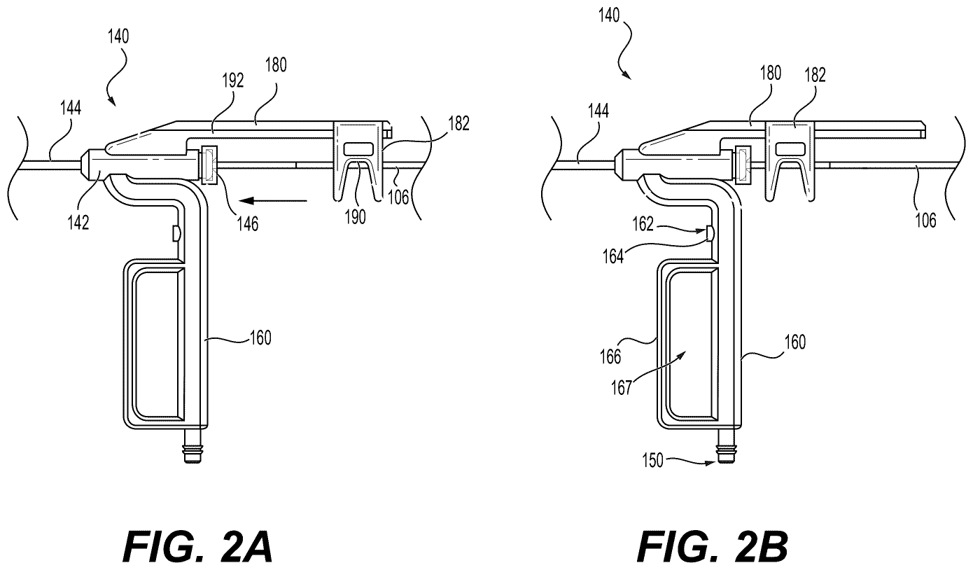

FIGS. 1-2B depict an exemplary medical system 100 according to the present disclosure. Medical system 100 includes a medical device 140 and optionally a scope 102 movable relative to the medical device. For example, medical device 140 may be configured to receive a portion of shaft 106 of scope 102.

Medical device 140 may include a handle 141 with a hub 142. Hub 142 may be generally cylindrical in shape as shown, however, this is merely exemplary. Hub 142 may have other shapes. Hub 142 may include a lumen with a proximal opening 146. The lumen of hub 142 may be sized and shaped to receive a shaft of a scope, e.g., shaft 106 of scope 102. For example, shaft 106 may be slidable in the proximal and distal direction within the lumen of hub 142. In some aspects, medical device 140 includes a sheath 144, e.g., coupled to hub 142 and extending distally from hub 142. Sheath 144 may be configured to receive a shaft of a scope therein, e.g., shaft 106 of scope 102.

Handle 141 may include a grip portion 160 configured to receive a palm of a user's hand. Grip portion 160 may extend transverse to the lumen of hub 142. In some aspects, grip portion 160 may extend substantially perpendicular to the lumen of hub 142, e.g., substantially perpendicular to a central longitudinal axis of hub 142. In some examples, handle 141 includes a suction channel in fluid communication with the lumen of hub 142 and with sheath 144, e.g., the channel being configured to provide suction to the lumen of hub 142 and to sheath 144. The suction channel may be configured to connect to a vacuum source. In some examples, the suction channel extends through grip portion 160. The suction channel may include a valve or adapter 150 at an end of the suction channel (e.g., at an end of grip portion 160) configured to connect to a vacuum source.

Handle 141 may include an aperture 162 that allows a user to control suction (vacuum) through the suction channel while the user grasps grip portion 160. For example, aperture 162 may be configured to vent the suction channel of handle 141 when aperture 162 is uncovered. When aperture 162 is covered (e.g., by a finger), vacuum in the suction channel may be greater (lower pressure and greater suction) than when aperture 162 is uncovered (higher pressure and less suction). Aperture 162 may be flush against an outer surface of handle 141 or may be defined through a protrusion 164 of handle 141 proximate grip portion 160. Grip portion 160 may be configured to receive a palm of the user's hand while a finger of the user's hand controls venting of aperture 162. Optionally, handle 141 may include an arch 166 that together with grip portion 160 defines an opening 167 to receive the user's hand as the palm of the user's hand grasps grip portion 160.

Handle 141 also may include an arm 180, e.g., extending proximally from hub 142. Arm 180 may extend along an axis substantially parallel to the lumen of hub 142, e.g., parallel to the central longitudinal axis of hub 142. Additionally or alternatively, arm 180 may extend transverse to the central longitudinal axis of hub 142. For example, arm 180 may be movable relative to hub 142 between a first configuration wherein arm 180 is substantially parallel to the lumen of hub 142 and a second configuration wherein arm 180 is transverse to the lumen of hub 142

Medical device 140 may include a member coupled to arm 180 and slidable relative to arm 180 (e.g., slidable along a length of arm 180). For example, the member may be slidable along a longitudinal axis of arm 180 between a proximal portion of arm 180 and a distal portion of arm 180. This example illustrates the member in the form of a cart 182. Cart 182 may include one or more projections 184, e.g., to facilitate a user's ability to slide cart 182 along arm 180. For example, as shown in FIGS. 1-3B, cart 182 may include four projections including a pair of proximal projections 184a and a pair of distal projections 184b. Cart 182 may have a generally rectangular shape with the projections 184a, 184b positioned in each corner. Cart 182 may include a recess 190 between proximal projections 184a and distal projections 184b. Recess 190 may be configured to receive a portion of the thumb or other finger of the user's hand to facilitate movement of shaft 106. According to some aspects, projections 184a, 184b may only include one projection, e.g., one proximal projection 184a or one distal projection 184b. Cart 182 may be configured to slide along arm 180 in a distal direction by a force applied to a proximal side of one or more of projections 184a, 184b and may be configured to slide in a proximal direction by a force applied to a distal side of one or more of projections 184a, 184b.

Arm 180 may include a rail 192 having a shape complementary to a channel 194 of cart 182 to permit cart 182 to slide along arm 180. For example, channel 194 may have a cross-section with a generally a “T”-like shape corresponding to a “T”-like shape of rail 192. In some examples, arm 180 may include a stop at or proximate a proximal end of rail 192, e.g., at or proximate a proximal end of arm 180. The stop may limit proximal movement of cart 182 along arm 180 so that cart 182 maintains its coupling to arm 180. For example, the stop may include a larger lateral dimension or a larger longitudinal dimension than channel 194 to prevent cart 182 from moving proximally beyond the stop.

According to some aspects of the present disclosure, medical device 140 may include a biasing element, such as a spring, configured to bias the member (e.g., cart 182) in a proximal or distal direction relative to arm 180. For example, the biasing element may bias cart 182 towards the proximal end of arm 180. An end of the biasing element may contact the distal end of arm 180 and another end of the biasing element may contact cart 182 (e.g., a distal portion of cart 182). In other examples, the biasing element may bias cart 182 towards the distal end of arm 180. An end of the biasing element may contact the stop at the proximal end of arm 180 and another end of biasing element may contact cart 182 (e.g., a proximal portion of cart 182).

The member, e.g., cart 182, may include features to facilitate using cart 182 to move a shaft along the lumen of hub 142. For example, the size and/or shape between proximal and distal protrusions 184a, 184b and/or the size and/or shape of recess 190 may correspond to the size and/or shape of shaft 106 of scope 102, e.g., to permit cart 182 and shaft 106 to move together relative to hub 142. In some examples, cart 182 may include surface features to increase friction with shaft 106 when a user's finger presses shaft 106 against cart 182.

Scope 102 as shown includes a handle 104 coupled to shaft 106. Scope 102 may include an actuator 112, for example, on handle 104, to facilitate articulation, steering, deflection and/or other movement of shaft 106. Shaft 106 may be flexible to facilitate navigation through tortuous anatomical passages or other cavities in a patient's body. Shaft 106 may define one or more lumens and may include or be coupled to a distal end portion that terminates in a distal tip. The one or more lumens may be in communication with corresponding lumens extending through handle 104 and/or a port 114. A user may insert an instrument or other device into port 114 and may extend the instrument or other device distally through the lumen of scope 102. Scope 102 may also include a light source and/or camera at a distal tip of shaft 106 and one or more actuators (in addition to actuator 112) to actuate the light source and/or camera. Handle 104 may be coupled to an umbilicus 120 connectable to one or more auxiliary devices. The one or more auxiliary devices may include a controller or control system, an imaging system, a power supply, a fluid supply, a suction/vacuum source, a display, etc.

Shaft 106 when inserted in medical device 140 may be coaxial with sheath 144. Shaft 106 may be slidable in the proximal and distal directions relative to sheath 144 and hub 142. For example, shaft 106 may be slidable along a length of sheath 144. Sheath 144 and shaft 106 may define a channel between an inner surface of sheath 144 and an outer surface of shaft 106 in communication with the suction channel of handle 141. Negative pressure provided to the suction channel of handle 141 may pull pressure through sheath and hub 142 through the suction channel. As discussed above, handle 141 may include an aperture 162 to permit the user to control the amount of suction provided by a vacuum source. Hub 142 may include features to form a seal with a shaft extending through the lumen of hub 142 while allowing the shaft to slide relative to hub 142. For example, the proximal opening 146 of hub 142 may be configured to form a seal around shaft 106. Proximal opening 146 may include an elastic material, e.g., O-ring. Proximal opening 146 may include a valve or adapter configured to form a seal with shaft 106 (e.g., a seal around an exterior surface of shaft 106).

Medical system 100 may be used in an exemplary medical procedure. For example, a user (e.g., medical professional) may insert sheath 144 of medical device 140 into a bodily lumen of a subject, such as through the urethra and/or ureter, to navigate the distal end of sheath 144 to a target site of the subject. The user may insert a trocar through hub 142 and sheath 144 in order to navigate sheath 144 to the target site. Once sheath 144 is proximate the target site, the user may withdraw the trocar and insert shaft 106 of scope 102 through hub 142 so that cart 182 is coupled to shaft 106. The user may move shaft 106 further in the distal direction by inserting the user's thumb into recess 190, pressing shaft 106 against cart 182, and then sliding cart 182 and shaft 106 together distally along rail 192. After reaching a desirable position of shaft 106 relative to the distal end of sheath 144, the user may stop moving cart 182 distally. If the user desires to further move shaft 106 distally or proximally relative to sheath 144, the user may slide cart 182 distally or proximally while again pressing shaft 106 against cart 182. Additionally or alternatively, the user may move shaft 106 solely with the user's fingers and/or with the user's other hand without the assistance of cart 182. The user may repeat these steps as necessary until a desired distance between the distal end of shaft 106 and the distal end of sheath 144 is achieved.

During a procedure, the user may control suction at a target site using medical device 140, e.g., provided by a vacuum source coupled to the suction channel of handle 141 and in fluid communication with sheath 144. For example, the user may grasp grip portion 160 with the user's palm and use a finger to close and/or open (vent) aperture 162 to increase or decrease suction, respectively, at the distal end of sheath 144. Suction may be used, for example, to capture materials including, but not limited to, fluids, kidney stones, dust, and/or other particles. Captured material(s) may be drawn in through sheath 144 via the channel radially outward of shaft 106 and travel proximally through the suction channel of handle 141 toward the vacuum source. To increase suction at the distal end of sheath 144, the user may cover or plug aperture 162 with a finger (e.g., an index finger or middle finger). When the user desired less suction the operator may uncover or unplug aperture 162. The user may repeat these steps as necessary during the procedure.

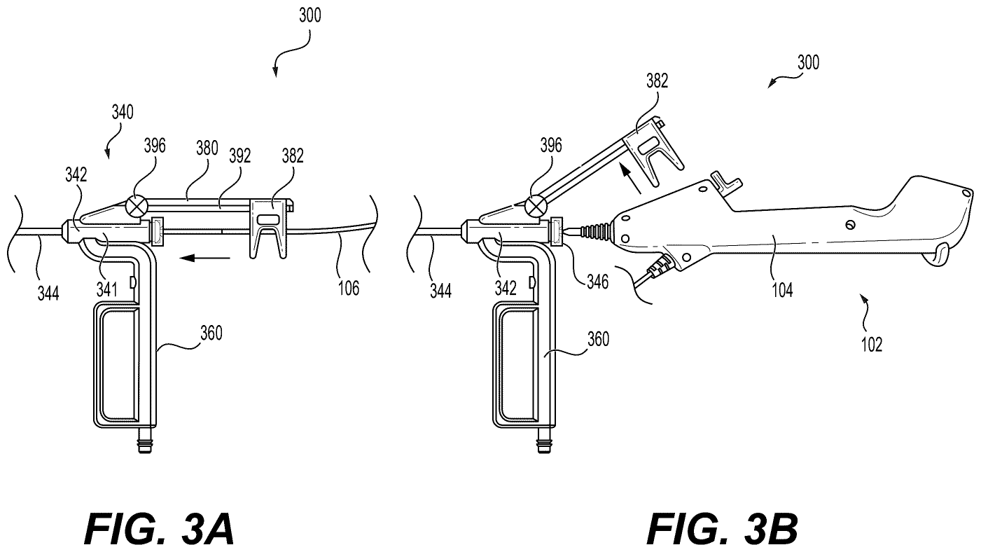

FIGS. 3A-3B depict another exemplary medical system 300 according to aspects of the present disclosure. Medical system 300 may include any of the features of medical system 100 discussed above unless otherwise specified. In this example, medical system 300 includes a medical device 340 and scope 102. Medical device 340 includes a handle 341 with a hub 342, a grip portion 360, and an arm 380. Arm 380 may extend proximally from hub 342, arm 380 being coupled to a member, e.g., cart 382, slidable relative to arm 380. For example, arm 380 may include a rail 392, wherein cart 382 is slidable along at least a portion of rail 392. Medical device 340 also includes a sheath 344 extending distally from hub 342, sheath 344 being in fluid communication with a suction channel of handle 341.

In this example, arm 380 is rotatable relative to hub 342, e.g., to allow a user to move arm 380 away from a central longitudinal axis of hub 342 and provide additional space proximate hub 342. The additional space may allow the user to move scope 102 in closer proximity to hub 342 (see, e.g., FIG. 3B), e.g., so that shaft 106 extends further distally relative to medical device 340. Arm 380 may be rotatable relative to hub 342 about a pivot 396 at a distal portion of arm 380. For example, arm 380 may include a first configuration wherein a longitudinal axis of arm 380 is substantially parallel to the central longitudinal axis of hub 342 (FIG. 3A), and a second configuration wherein the longitudinal axis of arm 380 is transverse to the central longitudinal axis of hub 342 (FIG. 3B). For example, arm 380 may be movable between a parallel position (an angle of 0 degrees relative to the central longitudinal axis of hub 342) to a transverse position of at an angle up to about 30 degrees, up to about 45 degrees, up to about 90 degrees, or even greater than 90 degrees, e.g., up to about 105 degrees or greater. As shown in FIG. 3B, transitioning from the first configuration to the second configuration of arm 380 may decouple cart 382 from shaft 106. When arm 380 is in the second configuration, handle 104 of scope 102 may be moved distally so that the distal end of handle 104 abuts a proximal opening 346 of hub 342.

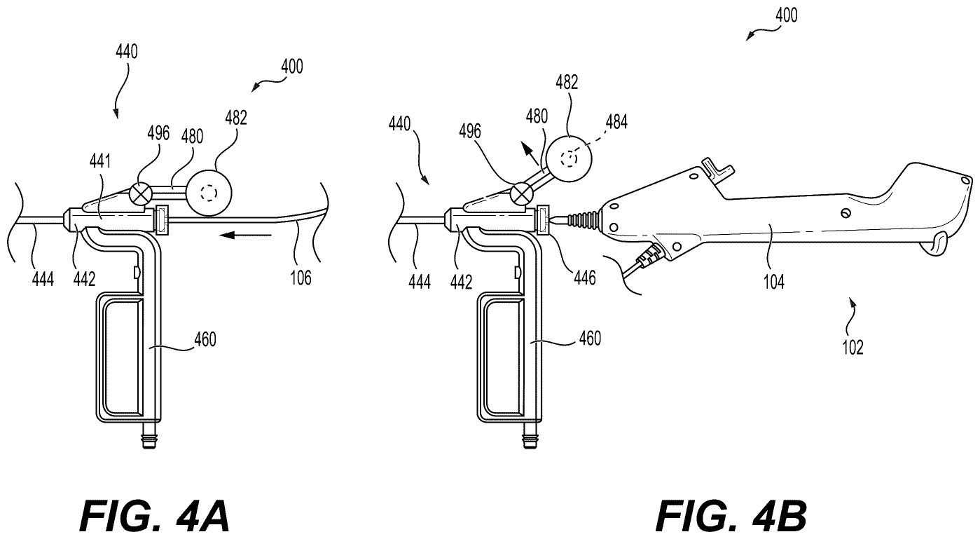

FIGS. 4A-4B depict another exemplary medical system 400 according to aspects of the present disclosure. Medical system 400 may include any of the features of medical system 100 and/or medical system 300 discussed above unless otherwise specified. In this example, medical system 400 includes a medical device 440 and scope 102.

Medical device 440 includes a handle 441 with a hub 442, a grip portion 460, and an arm 480. Arm 480 may extend proximally from hub 442, arm 480 being coupled to a member, e.g., wheel 482, rotatable relative to arm 480. For example, wheel 482 may be coupled to arm 480 via a pin 484 and rotatable about an axis defined by pin 484. In some examples, pin 484 may be at or proximate to a proximal end of arm 480. The rotation axis of pin 484 may be transverse, e.g., perpendicular, to a longitudinal axis of arm 480 and transverse to the central longitudinal axis of hub 442. Medical device 440 may be configured to receive a shaft, e.g., shaft 106 of scope 102, through a proximal opening 446 of hub 442 and through a sheath 444 coupled to handle 441.

Shaft 106 may contact an outermost curved surface of wheel 482. Shaft 106 may be slidable in the proximal and distal directions relative to hub 442 and sheath 444, e.g., slidable within the lumen of hub 442 and through sheath 444. A user may use wheel 482 to help slide shaft 106 relative to hub 442. For example, the user may press shaft 106 against wheel 482 using the thumb or other finger (e.g., shaft 106 being between the user's thumb or other finger and the surface of wheel 482) and move the thumb or other finger to rotate wheel 482 and move shaft 106. As shaft 106 slides in the proximal or distal direction, wheel 482 may rotate with shaft 106. According to some aspects, wheel 482 may be biased, e.g., via one or more biasing elements such as a spring, in a starting position before the user rotates wheel 482.

Optionally, arm 480 may be rotatable relative to hub 442, e.g., in a manner similar to arm 380 above, to permit scope 102 to move distally, closer to medical device 440, without interference by arm 480. For example, arm 480 may be rotatable relative to hub 442 about a pivot 496 at a distal portion of arm 480. For example, arm 480 may include a first configuration wherein a longitudinal axis of arm 480 is substantially parallel to a central longitudinal axis of hub 442 (FIG. 4A), and a second configuration wherein the longitudinal axis of arm 480 is transverse to the central longitudinal axis of hub 442 (FIG. 4B). Arm 480 may be movable between a parallel position (an angle of 0 degrees relative to the central longitudinal axis of hub 442) to a transverse position of an angle up to about 30 degrees, up to about 45 degrees, up to about 90 degrees, or even greater than 90 degrees, e.g., up to about 105 degrees or greater. In the first configuration, wheel 482 may contact shaft 106. To transition to the second configuration from the first configuration, the user may rotate arm 480 about pivot 496 so that wheel 482 decouples from shaft 106.

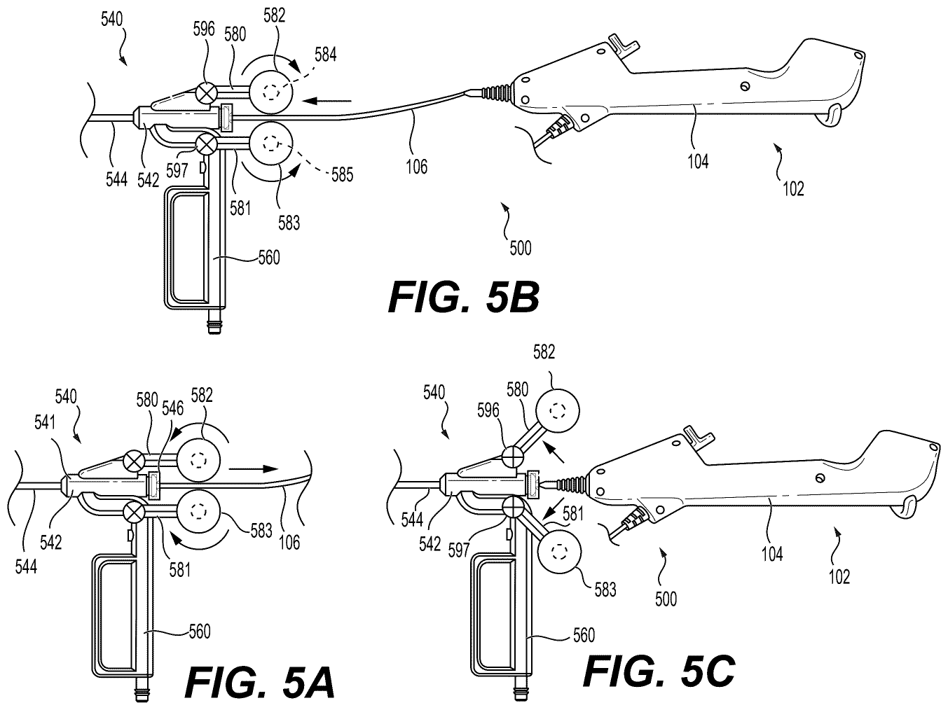

FIGS. 5A-5C depict another exemplary medical system 500 according to aspects of the present disclosure. Medical system 500 may include any of the features of medical system 100, medical system 300, and/or medical system 400 discussed above unless otherwise specified. In this example, medical system 500 includes a medical device 540 and scope 102.

Medical device 540 includes a handle 541 with a hub 542, a grip portion 560, and at least one arm, e.g., two arms 580, 581, extending proximally from hub 542. The longitudinal axis of first arm 580 may be substantially parallel to the longitudinal axis of second arm 581, e.g., first arm 580 being radially/circumferentially opposite second arm 581 relative to the central longitudinal axis of hub 542. Second arm 581 may be closer to grip portion 560 than first arm 580. Each arm 580, 581 may be coupled to a respective member, e.g., wheel 582 and wheel 583, rotatable relative to the arm 580, 581. For example, wheels 582, 583 may be coupled to the respective arm 580, 581 via a pin 584, 585 and rotatable about an axis defined by each respective pin 584, 585. In some examples, pins 584, 585 may be at or proximate a proximal end of the arms 580, 581. The rotation axes may be transverse, e.g., perpendicular, to a longitudinal axis of the respective arm 580, 581 and transverse to the central longitudinal axis of hub 542.

Medical device 540 may be configured to receive a shaft, e.g., shaft 106 of scope 102, through a proximal opening 546 of hub 542 and through a sheath 544 coupled to handle 541. Shaft 106 may be slidable relative to the central longitudinal axis of hub 542. Shaft 106 may contact an outermost curved surface of both wheels 582, 583, e.g., shaft 106 being between wheels 582, 583.

Shaft 106 may be slidable in the proximal and distal directions relative to hub 542 and sheath 544, e.g., slidable within the lumen of hub 542 and through sheath 544. A user may use wheels 582, 583 to help slide shaft 106 relative to hub 542. For example, the user may slide shaft 106 by contacting wheel 583 with the user's thumb or other finger to rotate wheel 583, which thereby moves shaft 106 along the central longitudinal axis of hub 542 and rotates wheel 582. Wheel 582 may help guide and/or align shaft 106 with the central longitudinal axis of hub 542. As shown in FIG. 5A, to slide shaft 106 proximally, the thumb or other finger of the user's hand may contact wheel 583 and rotate wheel 583 clockwise as wheel 582 rotates with shaft 106 in a counter-clockwise direction. As shown in FIG. 5B, to slide shaft 106 distally, the thumb or other finger of the user's hand may contact wheel 583 and rotate wheel 583 counter-clockwise as wheel 582 rotates with shaft 106 in a clockwise direction. According to some aspects, wheels 582,583 may be biased, e.g., via one or more biasing elements such as a spring, in a starting position before the user rotates wheel 482.

Optionally, arms 580, 581 may be rotatable relative to hub 542, e.g., in a manner similar to arm 480 above, to permit scope 102 to move distally, closer to medical device 540, without interference by arms 580, 581. For example, each arm 580, 581 may be rotatable relative to hub 542 about a respective pivot 596, 597 at distal portions of arms 580, 581. For example, each arm 580, 581 may have a first configuration wherein a longitudinal axis of arm 580, 581 is substantially parallel to a central longitudinal axis of hub 542 (FIGS. 5A and 5B), and a second configuration wherein the longitudinal axis of arm 580, 581 is transverse to the central longitudinal axis of hub 542 after rotating arms 580, 581 relative to hub 542 (FIG. 5C). In the first configuration of arms 580, 581, wheels 582, 583 may contact shaft 106 as discussed above. In the second configuration, each arm 580, 581 may be angularly displaced away from each other relative to the central longitudinal axis of hub 542. Each arm 580,581 may be movable between a parallel position (an angle of 0 degrees relative to the central longitudinal axis of hub 542) and at an angle up to about 30 degrees, up to about 45 degrees, up to about 90 degrees, or even greater than 90 degrees, e.g., up to about 105 degrees or greater.

To transition to the second configuration, the user may rotate arms 580, 581 about respective pivots 596, 597 and away from each other so that arms 580, 581 move away from the central longitudinal axis of hub 542. As shown in FIG. 5C, transitioning from the first configuration to the second configuration of arms 580, 581 may decouple wheels 582, 583 from shaft 106. In some examples, one of arms 580, 581 may be in the first configuration and the other may be in the second configuration, the arms 580, 581 being independently movable.

As mentioned above, one or both wheels 582, 583 may include a biasing element configured to return wheels 582, 583 to a predetermined position. The biasing element(s) may be configured to rotationally bias wheels 582, 583 in a direction to return shaft 106 to a starting position. In some examples, the biasing element of wheel 583 may be configured to rotationally bias wheel 583 in the clockwise direction, and the biasing element of wheel 582 may be configured to rotationally bias wheel 582 in the counter-clockwise direction. In another example, the biasing elements may be configured to rotationally bias wheel 583 in the counter-clockwise direction and wheel 582 in the clockwise direction.

While principles of this disclosure are described herein with reference to illustrative examples for particular uses, it should be understood that the disclosure is not limited thereto. Those having ordinary skill in the art and access to the teachings provided herein will recognize additional modifications, uses, examples, and substitution of equivalents all fall within the scope of this disclosure.

What is claimed is:

1. A medical device comprising:
a handle including:
a hub defining a lumen having a proximal opening;
a grip portion extending transverse to the lumen; and
an arm extending proximally from the hub, wherein the arm is coupled to a member slidable or rotatable relative to the arm;
wherein the grip portion is configured to receive a palm of a user's hand while a thumb of the user's hand controls sliding or rotation of the member.

2. The medical device of claim 1, wherein the member is a cart slidable along a length of the arm, or wherein the member is a wheel rotatable relative to the arm.

3. The medical device of claim 1, wherein the member is a cart that includes a projection, the cart being slidable along the arm in a distal direction by force applied to a proximal side of the projection and slidable in a proximal direction by force applied to a distal side of the projection.

4. The medical device of claim 3, wherein the arm includes a stop proximal to the cart that limits movement of the cart in the proximal direction.

5. The medical device of claim 3, wherein the arm includes a spring configured to bias the cart in the proximal direction.

6. The medical device of claim 1, wherein the member includes a wheel coupled to the arm.

7. The medical device of claim 6, wherein the arm is a first arm and the handle includes a second arm extending proximally from the hub and parallel to the first arm, wherein the wheel is a first wheel coupled to a proximal end of the first arm, and wherein the handle includes a second wheel coupled to a proximal end of the second arm.

8. The medical device of claim 1, wherein the arm is rotatable relative to the hub about a pivot at a distal portion of the arm.

9. The medical device of claim 1, further comprising a sheath extending distally from the hub, the sheath being in fluid communication with the lumen of the hub.

10. The medical device of claim 9, wherein the handle includes a suction channel in fluid communication with the sheath, an end of the suction channel being configured to connect to a vacuum source.

11. The medical device of claim 10, wherein the handle includes an aperture in communication with the suction channel, the aperture being configured to vent the suction channel.

12. The medical device of claim 10, wherein the suction channel extends through the grip portion.

13. The medical device of claim 9, further comprising a shaft extending through the lumen of the hub and through the sheath, the shaft being slidable along a length of the sheath.

14. The medical device of claim 13, wherein the hub includes a valve or an adapter configured to form a seal with the shaft.

15. The medical device of claim 13, wherein the shaft is a shaft of a scope.

16. A medical device comprising:
a handle including:
a hub defining a lumen that extends along a central longitudinal axis of the hub, the lumen having a proximal opening;
a grip portion extending transverse to the central longitudinal axis; and
an arm extending proximally from the hub, wherein the arm is coupled to a cart slidable along a length of the arm or wherein the arm is coupled to a wheel rotatable relative to the arm; and
a sheath extending distally from the hub, the sheath being in fluid communication with the lumen of the hub;
wherein the grip portion is configured to receive a palm of a user's hand while a thumb of the user's hand contacts the cart or the wheel.

17. The medical device of claim 16, wherein the handle includes a suction channel that extends through the grip portion, and an aperture configured to vent the suction channel, the suction channel being configured to connect to a vacuum source.

18. The medical device of claim 16, wherein the arm is rotatable relative to the hub about a pivot at a distal portion of the arm.

19. The medical device of claim 16, wherein the grip portion is substantially perpendicular to the central longitudinal axis of the hub.

20. A medical device comprising:
a handle including:
a hub defining a lumen extending along a central longitudinal axis of the hub and having a proximal opening configured to form a seal with a shaft;
a grip portion extending transverse to the lumen; and
an arm extending proximally from the hub and configured to pivot relative to the hub, wherein the arm is coupled to a member slidable along a length of the arm or rotatable relative to the arm; and
a sheath extending distally from the hub;
wherein the grip portion is configured to receive a palm of a user's hand while a thumb of the user's hand controls sliding or rotation of the member.
